(19) United States
(12) Plant Patent Application Publication
(10) Pub. No.: US 2014/0230108 P1
(43) Pub. Date: Aug. 14, 2014

(54) NECTARINE DENOMINATED 'ANDES NEC-1'

(50) ;

(71) Applicant: VIVEROS ASOCIADOS CHILE LTDA. (ANA Chile-Andes Nursery Association), (US)

(72) Inventor: Rodrigo Infante, Paine (CL)

(73) Assignee: VIVEROS ASOCIADOS CHILE LTDA. (ANA Chile-Andes Nursery Association), Paine (CL)

(21) Appl. No.: 13/766,209

(22) Filed: Feb. 13, 2013

(51) Int. Cl. A01H 5/08

(52) U.S. Cl. CPC A01H 5/0856
USPC PLT/201

(57) ABSTRACT

A new and distinct variety of clingstone nectarine tree denominated ‘Andes Nec-1’ that is similar to the nectarine ‘Venus’ in terms of its high postharvest potential, and firm in texture. The variety is distinguished from ‘Venus’ by producing fruit that is clingstone instead of freestone, has skin completely covered by red blush, and matures about two weeks earlier. The variety's harvest period is a week before the ‘Fantasy’ nectarine variety. Its fruit is solid red to red-purple blush over yellow flesh, with a round, symmetrical fruit shape.

### LATIN NAME

Prunus persica

### VARIETAL DENOMINATION

Andes Nec-1

### BACKGROUND AND SUMMARY

The present invention relates to the discovery and asexual propogation of a new variety of nectarine tree, Prunus persica denominated Andes Nec-1. This new cultivar resulted from controlled hybridization between the nectarine Venus (♀) and the nectarine genotype Pr-07-J (♂) performed in 2000 at Paine, Metropolitan Region, Chile (latitude −33°80′S., longitude −70°66′S., altitude 508 m above sea level)

### DISTINCTIVE CHARACTERISTICS OF THE VARIETY

Semi erect plant growth, medium vigor, with abundant flowering during mid season. Fruit round, symmetrical, yellow flesh, clingstone, blush red to red-purple 100% coverage and solid. Harvest period a week before Fantasy.

This variety's main feature is a slow pulp softening rate commencing during the last two weeks before the commercial harvest. This characteristic enables delaying the harvest, to increase the size of the fruit and soluble solids content without affecting their postharvest life potential. Secondly, it shows a high postharvest life potential, which can reach up to 50 days at 0° C. while maintaining its initial sensory quality. From a sensory point of view this is a variety of medium to high acidity with a balanced sweetness/acidity ratio and in general an average sensory quality. The variety is not susceptible to “chilling injury” and therefore can be used for exporting to distant markets from the production zone.

### BRIEF DESCRIPTION OF THE DRAWINGS

The drawings of the nectarine tree of the present variety are color photographs:

FIG. 1 shows typical specimens of the whole fruit in skin color and form.

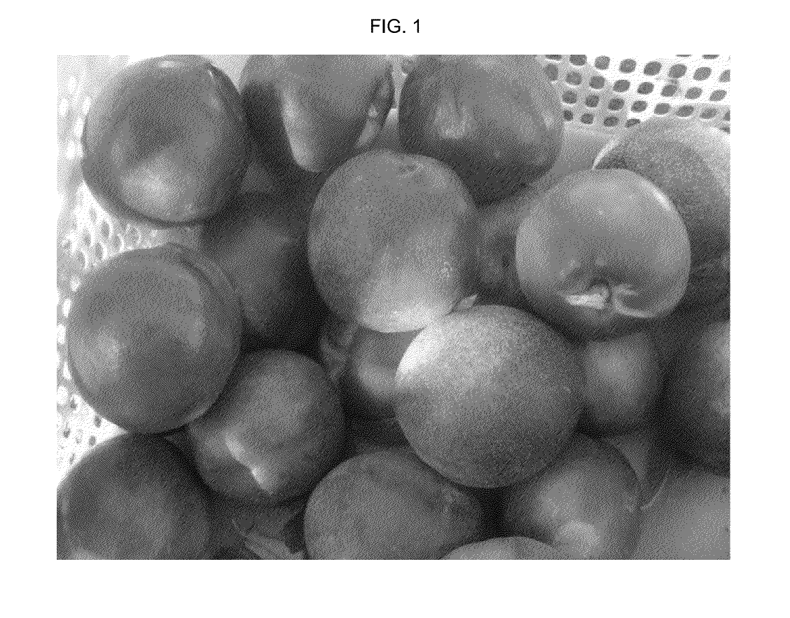

FIG. 2 shows typical specimens of the whole fruit, on tree, with typical leaves.

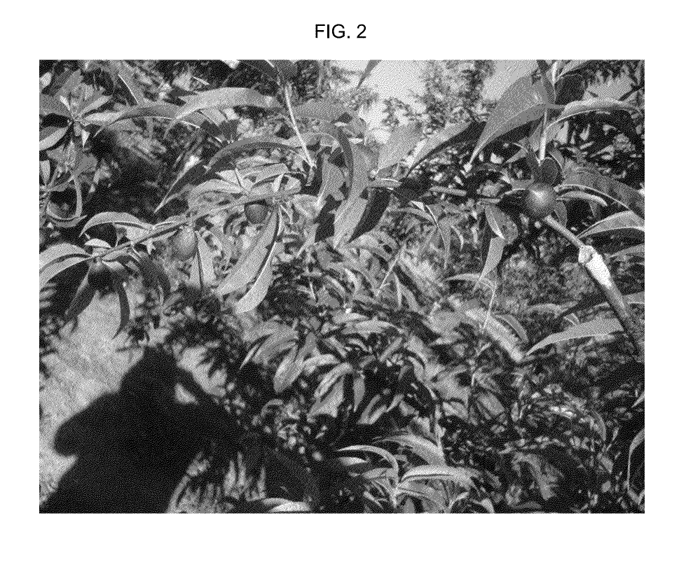

FIG. 3 shows a tree of ‘Andes Nec-1.’

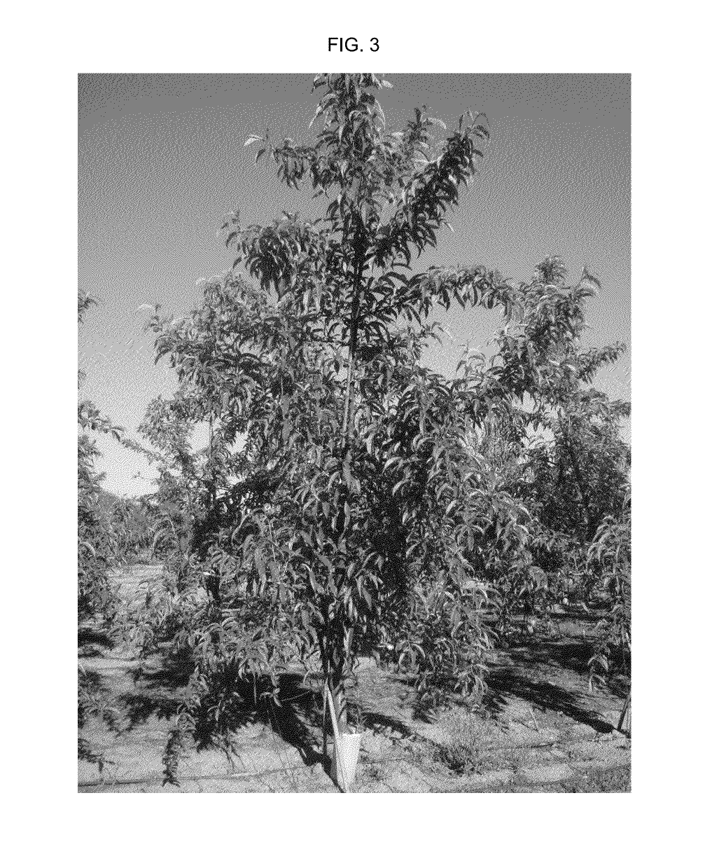

### DETAILED BOTANICAL DESCRIPTION

Tree:
   1. Size.—Medium, similar to Bonanza.
   2. Vigor.—Medium, similar to J.H. Hale.
   3. Habit.—Horizontal, like Albertina.
   4. Flowering shoot.—Thickness (excluding brindilles): Medium, similar to Redhaven.
   5. Flowering shoot.—Length of internodes: Medium, similar to Redhaven.
   6. Flowering shoot.—Anthocyanin coloration: Absent.
   7. Flowering shoot.—Intensity of anthocyanin coloration (as for 6): Weak, similar to Springtime.
   8. Flowering shoot.—Density of flower buds (as for 4): Medium, similar Michelini.
   9. Flowering shoot.—General distribution of flower buds (as for 4): In groups of two or more, similar to Redhaven.
Flower:
   10. Type.—Showy, similar to Robin.
   11. Calyx.—Color of inner side (opened flower, before falling of petals): Greenish yellow, similar to Robin.
   12. Corolla.—Predominant color (inner side): Medium pink, similar to Fuzalode.
   13. Petal.—Shape: Round, similar to Springtime.
   14. Petal.—Size: Medium, similar to Robin.
   15. Petals.—Number: Five, similar to Redhaven.
   16. Stamens.—Position compared to petals: Above, similar to Redhaven.
   17. Stigma.—Position compared to anthers: Same level similar to Crimson Gold.
   18. Anthers.—Pollen: present, similar to Redhaven.
   19. Ovary.—Pubescence: Absent, similar to Fuzalode.
   20. Young shoot.—Length of stipule (fully expanded leaf): Long, similar to Robin.
Leaf:
   21. Leaf blade.—Length: Long.
   22. Leaf blade.—Width: Medium.
   23. Leaf blade.—Ratio length/width: Medium, similar to Early Sungrand.
   24. Leaf blade.—: Shape in cross section: Flat, similar to Mayred.
   25. Leaf blade.—Recurvature of apex: Absent, similar to Merril Sundance.
   26. Leaf blade.—Angle at base: Acute, similar to Springtime.
   27. Leaf blade.—Angle at apex: Medium, similar to Earlyred.
   28. Leaf blade.—Color: Green, similar to Robin.
   29. Petiole.—Length: Short, similar to Redhaven.
   30. Petiole.—Nectaries present, similar to Redhaven.
   31. Petiole.—Shape of nectaries: Reniform, similar to Redhaven.
   32. Petiole.—Predominant number of nectarines: More than two, similar to Everts.
Fruit:
   33. Fruit.—Size: Medium, similar to Sunhaven.
   34. Fruit.—Shape (in ventral view): Round, similar to Redwing.
   35. Fruit.—Shape of pistil end: Plane, similar to Redhaven.
   36. Fruit.—Symmetry (viewed from pistil end): Symmetric, similar to Morettini.
   37. Fruit.—Prominence of suture: Weak, similar to Redhaven.
   38. Fruit.—Depth of stalk cavity: Shallow, similar to Robin.
   39. Fruit.—Width of stalk cavity: Medium.
   40. Fruit.—Ground color: Greenish yellow, similar to Precoce de Hale.
   41. Fruit.—Over color: Present.
   42. Fruit.—Hue of over color: Medium red, similar to Red Diamond.
   43. Fruit.—Pattern of over color: Solid flush, similar to Flavorcrest.
   44. Fruit.—Extent of over color: Large, similar to Suncrest.
   45. Fruit.—Pubescence: Absent.
   46. Fruit.—Density of pubescence: —.
   47. Fruit.—Thickness of skin: Medium, similar to Mme Girard.
   48. Fruit.—Adherence of skin to flesh: Strong, similar to Babygold5.
   49. Fruit.—Firmness of pulp: Firm, similar to Redhaven.
   50. Fruit.—Ground color of flesh: Yellow.
   51. Fruit.—Anthocyanin coloration directly under skin: Weakly expressed.
   52. Fruit.—Anthocyanin coloration of flesh: Absent or very weakly expressed, similar to Robin.
   53. Fruit.—Anthocyanin coloration around stone: Absent or very weakly expressed, similar to Springtime.
   54. Fruit.—Texture of the flesh: Not fibrous, similar to Redhaven.
   55. Fruit.—Sweetness: Medium, similar to Redhaven.
   56. Fruit.—acidity: Medium, similar to Redtop.
Stone:
   57. Stone.—Size compared to fruit: Medium similar to Redhaven.
   58. Stone.—Shape (in lateral view): Obovate, similar to Rubidoux.
   59. Stone.—Intensity of brown color Medium.
   60. Stone.—Relief of surface: similar to Madame Girerd.
   61. Stone.—Grooves, similar to Madame Girerd.
   62. Stone.—Tendency of splitting (at peak harvest): Absent or very low, similar to Fairhaven.
   63. Stone.—Adherence to flesh: Present, similar to Sweet Gold.
   64. Stone.—Degree of adherence to flesh: Strong, similar to Vivian.
Phenology:
   65. Time of leaf bud burst.—Early, similar to Springtime.
   66. Time of beginning of flowering.—Medium, similar to Redhaven.
   67. Duration of flowering.—Short, similar to Philp.
   68. Time of maturity for consumption.—Medium, similar to Fairhaven.
   69. Tendency to preharvest.—Absent, similar to Redhaven.
Fruit characterization:

| | | | | | | SOLU- |
|---|---|---|---|---|---|---|
| | | FIRMNESS (POUNDS) | | | | BLE |
| | BLUSH | Ecuatorial | | | | SOLID |
| SEASON | (%) | Diameter | Shoulders | Suture | Tip | (° Brix) |
| 2009-2010 | 90 | 13.9 | 12.2 | 14.0 | 13.2 | 13.5 |
| 2010-2011 | 90 | 12.2 | 11.2 | 12.3 | 12.4 | 13.2 |
| 2011-2012 | 98 | 12.2 | 11.8 | 11.7 | 11.6 | 11.2 |

### POSTHARVEST BEHAVIOR

At 50 days in cold storage +3 days at ambient temperature has good external appearance, no browning color or mealy texture, good taste and flesh firmness.

### GENERAL TECHNICAL NOTES

Very good outward appearance. Excellent color coverage. Fruit round, no tip. High productivity. Good flavor.

### CONCLUSIONS

After five years of evaluation, the variety continues to show its outstanding characteristics: very good productivity, good size, good flavor and good external appearance. High potential for export purposes because of exceptional post-harvest life.

Having thus described and illustrated the new variety of nectarine tree, I claim:

1. A new and distinct variety of nectarine tree, substantially as illustrated and described, that is similar to the nectarine ‘Venus’ in terms of its high postharvest potential, and firm in texture, but is distinguished therefrom by producing fruit that is clingstone instead of freestone in type, the skin is 100% covered by red blush, and that matures about two weeks earlier.
